(19) United States
(12) Patent Application Publication
(10) Pub. No.: US 2013/0338449 A1
(43) Pub. Date: Dec. 19, 2013

(54) REMOTE CONTINUOUS SEIZURE MONITOR AND ALARM

(71) Applicant: GLKK, Inc., Boerne, TX (US)

(72) Inventors: David G. Warwick, Austin, TX (US); Jeff Kane, Austin, TX (US); Mark Robert Lee, Austin, TX (US); Sean Kelley, Austin, TX (US); Thomas Lee Gardner, Boerne, TX (US)

(73) Assignee: GLKK, Inc., Boerne, TX (US)

(21) Appl. No.: 13/968,110

(22) Filed: Aug. 15, 2013

Related U.S. Application Data
(63) Continuation of application No. 13/100,185, filed on May 3, 2011, now abandoned.

(51) Int. Cl. A61B 5/0482

(52) U.S. Cl. CPC A61B 5/0482
USPC 600/301; 600/544; 600/545

(57) ABSTRACT

An electroencephalogram (EEG) utilizing epileptiform activity detection, warning and recording system adapted for use by non-healthcare professionals or healthcare professionals. The system is simple enough for use by untrained personnel and will be self-contained, not requiring technical setup or point of use maintenance. The system includes a small number of passive or active scalp electrodes for capturing the electrical signals in the subject's brain allowing for detection of epileptiform activity. The system will recognize epileptiform activity and will either transmit signals upon detection of epileptiform activity to either a localized warning device or to a cellular or radio receiving device. The system will note epileptiform activity in a recording component and allow for review of the epileptiform data to allow non-clinical and clinical personnel to verify such activity.

### CROSS REFERENCE TO RELATED APPLICATION

This application is a continuation application of U.S. application Ser. No. 13/100,185, filed May 3, 2011, now pending; which claims the benefit under 35 U.S.C. §119(e) of U.S. Ser. No. 61/330,806, filed May 3, 2010, the entire content of which is incorporated herein by reference in their entireties.

### FIELD OF THE INVENTION

The invention relates to the use of EEG (electroencephalography) based seizure/epileptiform activity detection, recording and alarm functions related to same.

### BACKGROUND OF THE INVENTION

The EEG is a long-standing technology that is based on brain wave recording. The detection of brain waves dates back to the late 19th century with the EEG being discovered in 1929, since that time the modern EEG has become a standard instrument common in hospitals since the early 1970's. In the last four decades the sophistication of EEG technology and the analysis of the recording has grown to become an industry mainstay in neurological diagnosis and detection of disease.

The industry focus up to the present time has been on detection of anomalous brain wave detection or epileptiform activity as associated with disease or brain activity abnormalities. EEG has been used extensively, as it has become more sophisticated, to specify seizures and to determine the location of the source of the seizure within the brain.

Ambulatory EEG (AEEG), which allows the patients to move with the portable apparatus while the EEG data are recorded continuously, has proven to be particularly useful for the following purposes: 1) to confirm a clinical suspicion of epilepsy; 2) to identify interictal epileptiform activity; 3) to document seizures that the patient is unaware of 4) to evaluate response to therapy; 5) to evaluate nocturnal or sleep-related events; 6) to evaluate suspected pseudoseizures; and 7) to evaluate syncope. However, like conventional EEG, human visual review of the vast amount of AEEG data has serious drawbacks.

Visual inspection is prohibitively time-consuming and inefficient. The raw data are typically voluminous and, even if the clinician reading the data is highly efficient, its comprehensive review and interpretation can be time consuming. Further, visual inspection lacks standards, leading to differing interpretations and missed characteristics in data.

Computer analysis (e.g., orthogonal transform (data segment averaging of wave and peak measurements), template matching (cross-correlation of a wave against pre-sets in a data template), inverse filtering (exclusion of white noise) and more sophisticated data analysis methods are helpful in overcoming limitations of human EEG data review. Because of their visual prominence during manual EEG interpretation, the automatic detection of epileptiform abnormalities might seem to be relatively simple. However, the complex nature of the EEG, its dependence on state of consciousness and activation, corruption by artifacts, and the wide biological variability both within and between patients renders even computer-assisted analysis of EEGs a task requiring a significant degree of training and sophistication for proper interpretation.

In human brain waves the electrical waveform is rhythmic and easily distinguished apart from the presence of significant brain injury or ‘brain death’. In a seizure, the brain wave is distinguishable from a ‘normal’ waveform by multiple atypical ‘features’ in the brain's activity. These ‘features’ can include wave amplitude, frequency, spiking, abnormal waveforms as well as others. These abnormalities allow a technician or doctor to detect a seizure and to recognize certain qualities of that seizure. The EEG typically is recorded on a storage device either by printing to a paper graph or electronically through software based memory systems. In every case to date, the technology requires that a trained professional analyze the EEG data. The analyst of the EEG may have to go through extended time periods of data thereby making the detection of abnormal events: labor intensive and expensive. In some newer technologies software and mathematical models are used as ‘feature extractors’ to highlight potential seizure events on the recorded data or to initiate the operation of other equipment such as video recorders but these target only the reduction of the time it takes the human technician to review the data or creating additional data.

Increasingly, EEG systems are moving toward possible prediction of the onset of a seizure through pre-seizure indicators. These predictive models are targeted at allowing either marking of likely seizure events or indicators on the EEG recording or marking of pre-seizure indicators or brain events that precede seizures for the purposes of diagnosis and warning. The goal of these devices is to predict, identify, specify and/or locate the source and the type of seizures for use of the data by healthcare professionals in disease management and treatment. As such, the EEG as used for epileptiform activity is exclusively a tool of the clinical professional with a few applications in biofeedback being explored outside of the clinical setting.

Additionally, non-clinical use of EEG technology is limited due to the size, cost and relative immobility of the EEG device. Typically, EEGs are performed in a hospital or clinic environment meaning that the patient cannot continue their normal daily activities and must be present in the hospital/clinic where the EEG is located. While ambulatory EEG devices have been developed they still require an array of sensors that are cumbersome to install and maintain and can be intrusive when used by the patient.

Such ‘portable’ devices can usually only maintain data gathering for an average duration of 48-72 hours before they must be returned to the medical facility for data transfer and analysis. This does not allow some seizures, or other abnormalities, which may or may not have occurred in this short time window, to be detected. If a patient suffers from irregular or infrequent abnormal brain activity the possibility of the EEG missing the event is fairly high.

Thus, EEG use has typically involved expensive, cumbersome, time limited, and/or analysis intensive devices that serve the needs of the doctor but are not focused on patient comfort or use by the consumer or non-clinician. A need, therefore, remains for an ambulatory seizure detection device that is portable, simple to use, and allows the sufferer and their caregivers to quickly assess if a seizure has occurred without interpretation of EEG data.

### SUMMARY OF THE INVENTION

The invention provides a portable or ambulatory seizure detection device which detects if a seizure is occurring in a particular time window and provides feedback regarding such brain wave activity in a simple and reliable format which does not require special training to interpret.

The device detects if probable epileptiform activity is occurring, records and warns, in binary fashion (alarm or not) an interested party of this probability. In response to the alarm, a caregiver can provide observation and attention to the seizure sufferer in the event that serious side effects occur. No data are reported (other than the alarm indicative of a probable seizure event) nor displayed (thereby reducing the necessary footprint of the device). Set up and use of the device requires little more placing the detection headgear on the person to be monitored and turning the monitoring system on. As such, anyone can use the system, even someone with no medical training or who has limited comfort with electronic devices.

To that end, the invention provides a simplified EEG system with relatively few EEG electrodes and other sensors, simplified function and analysis, comfortable headgear resulting in limited interference in the patient's daily routine. As such, the present invention provides a system for monitoring and reporting a threshold level of epileptiform activity in the brain of a subject. In one embodiment, the system includes: a) a headset comprising a plurality of EEG electrodes for collecting electrical activity from the brain of the subject; b) a base unit in communication with the plurality of electrodes; and c) an alarm output in communication with the base unit for receiving the alarm signal and generating an alarm. To minimize the footprint of the base unit, it preferably lacks any externally readable display except, optionally, a light which turns on or flashes to signal an alarm. In a preferred embodiment, the base unit is less than 2 square feet in length and width. In a particularly preferred embodiment, the base unit may be a portable data processing unit, such as a cell phone or other personal digital assistant (PDA).

The base unit includes a processor for generating records of epileptiform activity and a memory from storage of the records. In various embodiments, the processor includes computer-executable instructions for: (i) analyzing a digital input generated from electrical signals collected by the plurality of EEG electrodes to identifying whether a predetermined epileptiform activity level has been exceeded; (ii) generating an alarm signal when the predetermined epileptiform activity level has been exceeded; and (iii) time stamping and storing the digital signal when an alarm signal has been generated. Most preferably, the record does not include unprocessed or raw data of electrical brain activity, but rather a simple binary (yes/no) indication of whether the predetermined epileptiform activity level was exceeded.

According to another embodiment of the invention, the invention provides a simplified EEG system utilizing multiple indicators to confirm the possibility of epileptiform activity and therefore increase the accuracy of the device to the degree that the most likely anomalous events are readily identified. These indicators may include other physiological data captured by sensors mounted on the headset or elsewhere on the body, such as but not limited to ECG, biomarkers, temperature, motion, heart rate, humidity, breathing rate, blood gas concentration, and the like, used in conjunction with EEG readings.

According to another embodiment of the invention, the invention provides a simplified EEG system, preferably utilizing up to 12, and preferably less than 12, most preferably two to four sensors wired or wirelessly connected to a modular EEG with algorithms and software in place that will use multiple feature extractors to determine if a seizure may be occurring and send a wired or wireless signal to an alarm device thereby providing a warning system of ongoing or recent epileptiform activity.

According to another embodiment of the invention, the invention provides a simplified EEG system that detects epileptiform activity and stores these events to a computer-readable memory module for review and recording purposes for clinicians. Clinicians can then use this information to modify or alter therapeutic programs for the patient.

According to another embodiment of the invention, the invention provides a simplified EEG system that detects epileptiform activity and records these events for review and recording purposes for the support of FDA mandated clinical studies. Providers of therapeutic devices or drugs can utilize the system to confirm the efficacy of their target therapy during FDA mandated clinical studies. These studies can utilize data from the system to support filing and modify drug or device use criteria in early stage trials or dosing investigations.

According to another embodiment of the invention, the invention provides a simplified EEG device that detects epileptiform activity and records these events through EEG electrodes and other sensors that are applied to the patient's cranium with a minimal amount of discomfort. This may be achieved with an electrode head apparatus (EHA) or some other comfortable arrangement, which will house the sensors, the wires, and in the case of the wireless form of the device, a battery pack and transmitter.

According to another embodiment of the invention, the invention provides a simplified EEG system that detects epileptiform activity and records these events with a base unit system that is in close proximity to the patient to allow a hardwire connection or within the signal strength radius of a wireless transmitter system. It will contain a sensor signal receiver, a logic board which will filter, strengthen and process the signal, the algorithms designed to decipher the signal, and the software to operate the EEG processor and warning device.

According to another embodiment of the invention, the invention provides a simplified EEG system that detects epileptiform activity and records these events and includes a transmitter that will send a wired or wireless signal to a remote alarm device that will be as simple as a flashing light or audible alarm or as complex as a cellular phone or computer application that will receive the signal and initiate a warning protocol.

According to another embodiment of the invention, the invention provides a simplified EEG system that detects epileptiform activity and records these events and the system contains any of a number of validating devices and sensors that will increase the accuracy of seizure detection through confirmatory parameters. Such validating devices and sensors include accelerometers to indicate movement of the patient either in the device headgear or separate from the device, ECG sensors, pulse oximeter, or other physiological indication devices also coupled with the device for confirmatory indications of seizure.

The invention further provides a method for monitoring and reporting a threshold level of epileptiform activity in the brain of a subject by producing a simply interpreted report of epileptiform activity. The method includes: a) collecting electrical activity from the brain of the subject via a headset comprising a plurality of EEG electrodes; b) analyzing a digital input generated from electrical signals collected by the plurality of EEG electrodes to identifying whether a predetermined epileptiform activity level has been exceeded; c) generating an alarm signal when the predetermined epileptiform activity level has been exceeded; d) time stamping and storing the digital signal to a data storage device when an alarm signal has been generated, thereby producing a record; and e) transmitting the alarm signal to an alarm output to generate an alarm. In various embodiments, the record does not include unprocessed or raw data of brain activity so that the record may be easily interpreted by an untrained individual.

### BRIEF DESCRIPTION OF THE DRAWINGS

Having thus described the presently disclosed subject matter in general terms, reference will now be made to the accompanying Figures, which are not necessarily drawn to scale, and wherein:

FIG. 1 depicts a EHA cap design;

FIG. 2 depicts an alternative EHA cap design with EEG electrodes and additional sensors;

FIG. 3 depicts a EHA headband design;

FIG. 4 depicts a schematic layout of the system of the invention;

FIG. 5 depicts a schematic layout of the firmware program utilized to govern the functionality of the system of the invention;

FIG. 6 depicts a workflow process for operation of the firmware.

### DETAILED DESCRIPTION OF THE INVENTION

Electroencephalogram (EEG) waveforms contain information that can be used in the detection of seizure events. An EEG signal can be examined in a temporal context, in which the amplitude is measured against time or the spectral context, where the signal is examined as power measured against frequency. Most cerebral signals recorded are typically between 0.5 Hz and 100 Hz, but EEG devices record a much larger range between 0.5 and 200 Hz. When digitized, characteristics of neurological signals can be processed by learning algorithms, statistical methods or digital signal analysis to ascertain when a seizure occurs, for how long it occurs and in which frequency range it occurs. Before any determination can be made about the presence of seizures, digital filters are used to generate distinct features of an EEG waveform that are unique to the subject being observed.

The invention provides a simplified electroencephalogram (EEG) system that uses a smaller number of passive or active scalp EEG electrodes for capturing the electrical signals in the subject's brain than is common in ambulatory or clinical EEG devices for the purpose of detecting epileptiform activity or other neurological events and characteristics associated with, but not limited to the following: sleep disorders, schizophrenia, cognitive neuroresponses, autism, behavior disorders, epilepsy, narcolepsy, neurological and psychiatric disorders such as cerebral deficits subsequent to cardiac bypass surgery and grafting, stroke, ischemic stroke, cerebral ischemia, spinal cord trauma, head trauma, perinatal hypoxia, cardiac arrest, hypoglycemic neuronal damage; Huntington's Chorea; amyotrophic lateral sclerosis; multiple sclerosis; ocular damage; retinopathy; cognitive disorders; idiopathic and drug-induced Parkinson's disease; muscular spasms and disorders associated with muscular spasticity including tremors, epilepsy, convulsions; cognitive disorders including dementia (associated with Alzheimer's disease, ischemia, trauma, vascular problems or stroke, HIV disease, Parkinson's disease, Huntington's disease, Pick's disease, Creutzfeldt-Jacob disease, perinatal hypoxia, other general medical conditions or substance abuse); delirium, amnestic disorders or age related cognitive decline; schizophrenia or psychosis including schizophrenia (paranoid, disorganized, catatonic or undifferentiated), schizophreniform disorder, schizoaffective disorder, delusional disorder, brief psychotic disorder, shared psychotic disorder, psychotic disorder due to a general medical condition and substance-induced psychotic disorder; substance-related disorders and addictive behaviors (including substance-induced delirium, persisting dementia, persisting amnestic disorder, psychotic disorder or anxiety disorder; tolerance, dependence or withdrawal from substances including alcohol, amphetamines, cannabis, cocaine, hallucinogens, inhalants, nicotine, opioids, phencyclidine, sedatives, hypnotics or anxiolytics); movement disorders, including akinesias and akinetic-rigid syndromes (including Parkinson's disease, drug-induced parkinsonism, postencephalitic parkinsonism, progressive supranuclear palsy, multiple system atrophy, corticobasal degeneration, parkinsonism-ALS dementia complex and basal ganglia calcification), chronic fatigue syndrome, fatigue, including Parkinson's fatigue, multiple sclerosis fatigue, fatigue caused by a sleep disorder or a circadian rhythm disorder, medication-induced parkinsonism (such as neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremor), Gilles de Ia Tourette's syndrome, seizure disorders, epilepsy, and dyskinesias including tremor (such as rest tremor, essential tremor, postural tremor and intention tremor), chorea (such as Sydenham's chorea, Huntington's disease, benign hereditary chorea, neuroacanthocytosis, symptomatic chorea, drug-induced chorea and hemiballism), myoclonus (including generalised myoclonus and focal myoclonus), tics (including simple tics, complex tics and symptomatic tics), restless leg syndrome and dystonia (including generalised dystonia such as iodiopathic dystonia, drug-induced dystonia, symptomatic dystonia and paroxymal dystonia, and focal dystonia such as blepharospasm, oromandibular dystonia, spasmodic dysphonia, spasmodic torticollis, axial dystonia, dystonic writer's cramp and hemiplegic dystonia); attention deficit/hyperactivity disorder (ADHD); conduct disorder; migraine (including migraine headache), and the like.

The system also has an alarm responsive thereto warn the patient or a caregiver of detected seizure activity. The system digitizes the captured electrical signals from the brain and implements at least one algorithm that analyzes the waveforms to detect patterns or features in the signals that indicate a seizure occurred. Based on detection of epileptiform activity exceeding a pre-determined threshold, the device provides an alarm to a base unit detection device, a remote monitor (e.g., used by a caregiver at a location other than where the patient is present), or both. Given the simplicity of the system and its suitability for use in overnight monitoring, the system is particularly well-suited for use in monitoring seizure activity in children, invalids or sleeping individuals.

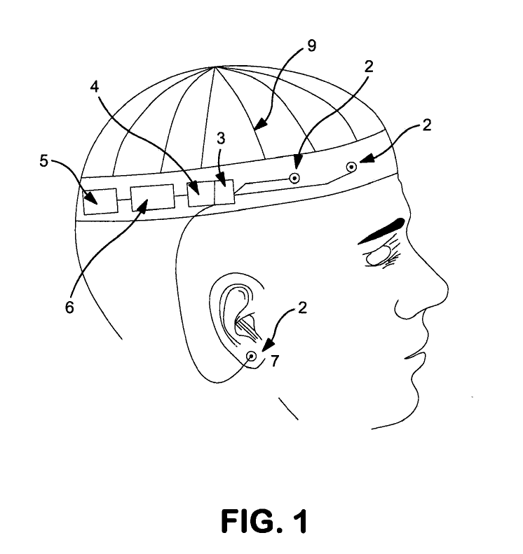

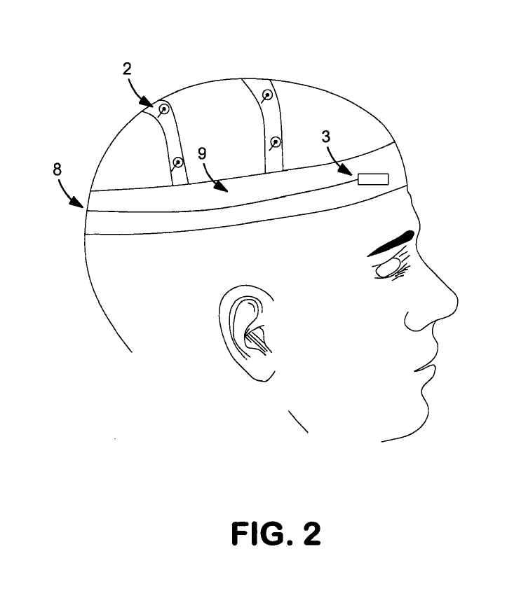

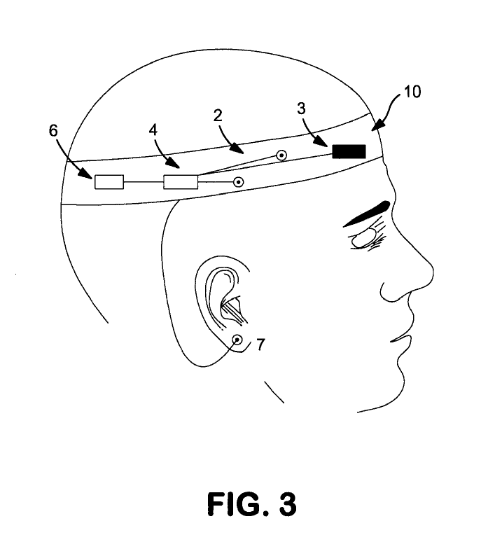

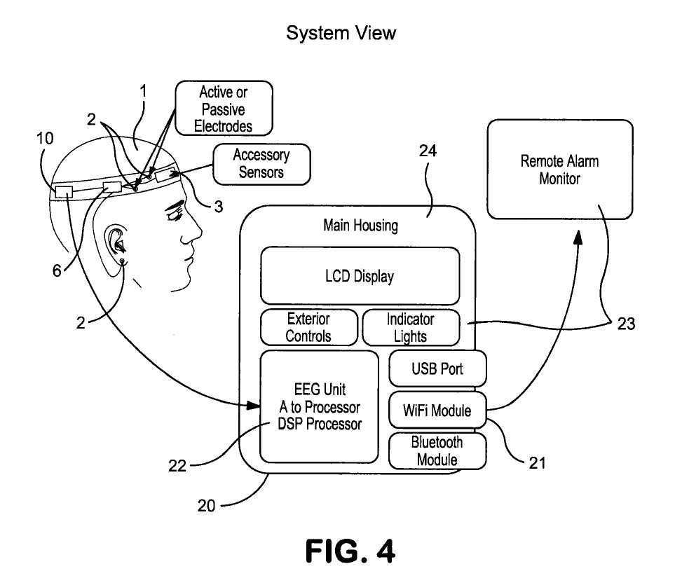

The system is divided into three main mutually dependent sub-assemblies: the electrode head apparatus (EHA) (which may be in any convenient form, such as the cap design of FIGS. 1 and 2 the headband design of FIG. 3); the base unit and housing, shown in FIG. 4 (system with housing), including a remote alarm unit that will receive signals using, for example, a wired connection for communication of such signals or a wireless one, such as a WiFi or Bluetooth™ protocol, or other wireless signal, to warn a subject's caregiver that a seizure event has occurred or is occurring.

As shown in FIGS. 1 and 2, EHA 1 is placed on the subject's head to bring amplified active electrodes or unamplified passive electroencephalogram (EEG) electrodes 2 in contact with the subject's scalp. Conveniently for the user, electrodes 2 may be provided pre-attached to EHA 1 to dispose in contact with clinically desirable sites on the subject's head when EHA 1 is placed thereon to detect waveforms in the brain; e.g., along the circumference of EHA 1 around the subject's head (FIG. 1) and/or along its crown (FIG. 2).

Given the limited scope of data captured by the system of the invention, 12 or fewer EEG electrodes are provided, preferably 2 to 8 EEG electrodes, and more preferably 4 to 6 EEG electrodes, and most preferably 3 EEG electrodes (in comparison to the 20 or more EEG electrodes required by commercially available EEG caps). More particularly, the system of the inventions designed to operate with a minimum of two channels, with at least three EEG electrodes 2 placed on the subject's head and a “driver right leg” (DRL) input electrode 7 on the earlobe or other area near the ear.

In a typical two channel configuration, a pair of electrodes is placed on one side the subject's head to detect signals in a single hemisphere of the brain. In a differential mode, each channel of the AD captures a signal that is an amplified voltage difference of two electrodes placed on two different parts of the same hemisphere of the subject's head. For example, two electrodes can be placed on the frontal and parietal areas of each hemisphere for a total of two input channels. The exact location of the electrodes on each hemisphere will differ among patients because of a lack of uniformity in seizure activity. An EEG with two channels, for example, may have two electrodes on each hemisphere of the head. The EEG can also be configured for a common reference configuration, allowing for one common reference electrode shared by each channel and two additional electrodes acting as a channel input for each channel.

Optionally, accessory sensors 3 (FIG. 2), such as accelerometers and piezoelectric gyroscopes to determine head orientation and movement (especially useful in monitoring physical movements in a subject that may accompany seizure) and position to provide additional data to the input of the processor contained in the main housing, may be provided on EHA 1. Sensors 3 and electrodes 2 may be any conventional design adapted for collecting EEG signals. As noted and shown in FIGS. 1 and 3, the system may also utilize a DRL electrode 7 that will be placed on or near the ear to act as a common mode feedback that is sent to the subject's body in order to reduce artifacts in the EEG signal commonly caused by nearby 60 Hz noise from A/C power wiring, lighting or nearby devices connected to an A/C power source.

For wireless transmission of signals from EEG electrode array 2 and optional sensors 3 to the base unit (FIG. 4), wireless transmitter 6 is also pre-attached to EHA 1, as shown in FIG. 1. Alternatively, as shown in FIG. 2, wired connector 8 may be provided for hard attachment to the base unit. In either case, electrode substrate 9 of EHA 1 may conveniently be made of any conventional material for use as electrode substrates EEG devices and will preferably have elastic properties (such as biocompatible polymers) to allow EHA 1 to be universally sized to fit subjects with heads of differing sizes.

EEG electrodes 2 will transmit signals to AD (analog-to-digital) converter 22 (FIG. 4) by a wire or by a standard wireless data transmission such as Bluetooth or WiFi. If EHA 1 is wireless, wireless module 6 will be on board to facilitate signal transmission and low profile rechargeable batteries 5 (FIG. 1) will preferably be provided.

In an alternative embodiment which may provide additional user comfort, EHA 1 may be a headband 10 fittable around the subject's head.

Turning to FIG. 4, system 20 includes wireless receiver 21 (e.g., for use with WiFi, Bluetooth or other data transfer protocols) which receives signals from AD input or integrated circuit 4 (see, e.g., FIGS. 1 and 3) collected from electrodes 2. The signals are simultaneously read and recorded by processer 22. Signals collected from the electrodes and can be set to record a minimum of 1 and up to 12 channels of data, preferably less than 12 and most preferably 2 to 4 channels, on a storage device such as a hard drive or an industry standard flash memory system. The subject's brain may produce signals of differing frequency at any given moment, and the system will collect signals of interest in the range of 0.5 Hz to 200 Hz. The data channels can be recorded as the voltage difference between two electrodes placed on the head or as the voltage difference between an electrode and another common electrode shared between all channels.

More particularly, computational and support electronics resident in system 20 may further include, but are not limited to, RAM memory, flash or drive storage, and a display to show the operational status of the device and small waveforms to show the EEG signals. The system can be adapted to connect the EEG to a computer by means of a standard USB port in order to update software and to allow a physician to extract EEG data from the unit for review.

The software that detects epileptiform activity or other neurological events on the System utilizes, but is not limited to, learning algorithms, digital signal processing and statistical methods to determine the parts of an EEG waveform that contain seizure or other neurological activity. Software learning algorithms determine the probability of a seizure occurrence in recorded EEG data by comparing features of newly recorded data to that of EEG data containing both non-seizure events and seizure events.

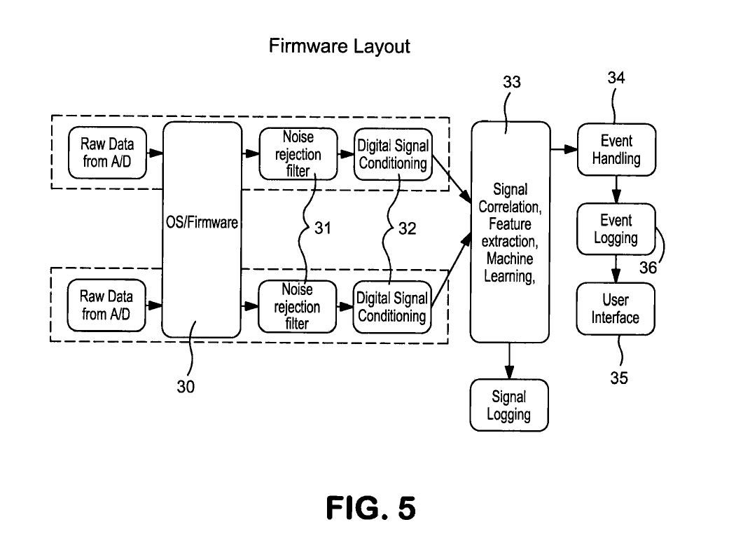

As shown in FIG. 5, software (firmware) 30 filters noise in the signal received from each EEG electrode (in filtering module 31) and further conditions the data (in conditioning module 32) to determine if the signal exceeds a pre-determined threshold. The conditioning is performed in an automated process that examines the stored EEG data to characterize the data features for learning algorithms (if resident in signal correlation module 33), annotates them if necessary, and determines if the data contains waveform elements indicative of a seizure.

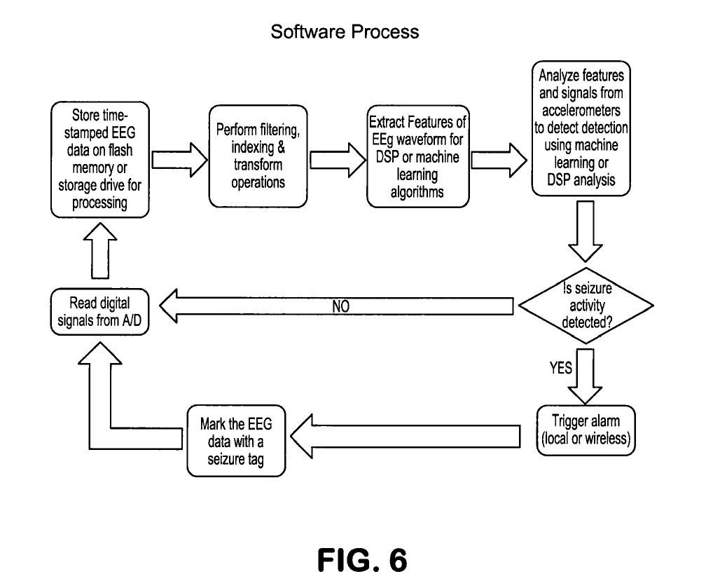

More particularly, as shown in FIG. 6, the filtering program (resident on filtering module 31 of FIG. 5) rejects unnecessary high frequency signals or noise, then through electrostatic protection and a current limiter to prevent damage to the monitor. These filters are placed before and after amplification to reduce signal artifacts, after which the signal is passed to the AD (or, for use with more than two channels, more than one AD) for converting to a digital signal. Other arrangements of the amplifiers can allow for one EEG electrode per channel if each channel shares an electrode to be used as a common signal.

The differential amplifier increases the strength of the electrical signals. This signal is further filtered electronically to remove undesirable background noise (such as from involuntary movements during sleep) with a notch filter after the signal has been amplified. The filtered signal then passes through an analog to digital converter to convert the captured signals into a digital form. The signal can then be further filtered by digital signal processing techniques using a microprocessor.

The system can monitor one or more EEG channels, utilizing digital signal processing techniques such as a Fast Fourier Transform (FFT) and other algorithms to extract spectral information from an EEG waveform. The learning algorithm component of the software compares both temporal and spectral features of the EEG waveform to that of a waveform of known seizure activity to determine whether a seizure has occurred. The determination of features of waveform data is based on comparing new EEG waveform data to data sets that contain seizure and non-seizure events.

Any portion of an EEG signal that was not produced in the brain is considered an artifact and can possibly degrade the accurate detection of a seizure. Artifacts that can be detected by an EEG include muscle, tongue and eye movements; poor electrode contact on the scalp; electromagnetic interference; and movement. Artifacts improperly labeled as brain activity can contribute to the false determination of seizure activity. As part of the software process to detect seizures, feature extraction may be performed to reject artifacts in signal correlation module 33 (FIG. 5), and algorithms for “machine learning” to refine seizure detection by excluding non-seizure events registered over time may also be provided.

The learning algorithms that detect a seizure construct a collection of features of the waveform for comparison to the features of data with known epileptiform activity. Some features of an EEG waveform important for seizure detection include spikes, number of peaks and valleys in a signal, mean amplitude of the signal, the absolute value of signal data, rhythmic bursts or spindles. Features of an EEG are collected into a feature vector, which are used by learning algorithms as a support vector machine, a process that builds a model based on training data to recognize classifications of data. Using learning algorithms to compare new EEG data to EEG data that contains known events minimizes the risk for false positives in seizure detection. The ultimate goal of determining features is to classify waveform activity as either that of a seizure of that of normal brain activity.

After the waveforms collected from the subject's brain are converted by AD processor 22, the processor runs software (FIG. 5) to timestamp and store the data on flash memory or a storage drive, perform any necessary digital filtering, and transform the data to its spectral components. In the case that machine learning algorithms are used for seizure detection, the software will examine the data and extract features of the spectral and temporal components of the signal. All of the information derived from the previous operations is used to determine the existence of a seizure, and appropriate alerts are initiated.

The data is examined by the system in epochs, or signal data windows with a specific duration, usually on the order of 2 to 3 seconds. Where a learning algorithm(s) is used, the signal is analyzed to extract core features for machine training. A common implementation of machine learning is the use of a Support Vector Machine (SVM), a software process that builds on training the learning system to recognize classifications of data of a patent for use in future event detection. When an epileptiform event is accurately detected, it increases the probability that a true seizure event will be detected in the future

As reflected in the flowchart of FIG. 6, if a seizure is detected by correlating the filtered and conditioned signal to a pre-determined triggering threshold (e.g., by comparing the signal to data sets based on previously detected epochs at signal correlation module 33), an alarm is triggered in event handling module 34 that is conveyed via user interface 35, and optionally recorded for later review at event logging module 36. The alarm 23 (FIG. 4) may be a visual one (e.g., warning light), an audible noise (conveyed through a speaker in housing 24 of system 20; FIG. 4), a physical (e.g., vibratory) alarm or a small display (e.g., shown on an LCD screen). Alarm 23 may be provided in housing 24 or, optionally, on a remote monitor whereby the alarm is triggered by wired or wireless transmission of a seizure detection signal to a dedicated monitoring device or a non-dedicated one, such as a cellular phone or PDA.

For the user, instructions are provided regarding placement of EHA 1 on the subject's head, synchronization with the wireless module in system 20 (if present), and operation of the base unit (e.g., by turning it on). Although software updates may be obtained, no additional software installation or hardware set up will be required. Suggestions for appropriate responses to alarms may also be provided in user instructions, such as conditions which indicate necessity for the services of a physician or emergency assistance to be sought.

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

The subject treated by the presently disclosed methods and devices in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein.

The term “effective,” as that term is used in the specification and/or claims, means adequate to accomplish a desired, expected, or intended result, e.g., to prevent, alleviate, or ameliorate symptoms of disease or prolong the survival of the subject being treated.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

All publications, patent applications, patents, and other references are herein incorporated by reference to the same extent as if each publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.

The invention having been described by reference to particular embodiments, those of ordinary skill in the art will recognize that modifications or adaptations of the invention to particular uses are possible and within the scope of the invention. The latter is defined by the appended claim(s).

What is claimed is:

1. A system for monitoring and providing an alarm indicating that a threshold level of epileptiform activity has been exceeded in the brain of a subject, comprising:
a) a headset comprising from 1 to 12 electroencephalogram (EEG) electrodes for collecting electrical activity from the brain of the subject;
b) a portable base unit in communication with the plurality of EEG electrodes, the base unit comprising:
a processor comprising computer-executable instructions for acting on data received from said plurality of EEG electrodes, the actions consisting essentially of:
(i) filtering, conditioning and analyzing a digital input generated from electrical signals collected by the plurality of EEG electrodes and transmitted from the headset to identify whether a predetermined epileptiform activity level has been exceeded;
(ii) generating an alarm signal when the predetermined epileptiform activity level has been exceeded;
(iii) time stamping and storing the digital signal when an alarm signal has been generated, thereby producing a record, wherein the record does not include unprocessed data of electrical brain activity; and
a memory for storage of the record; and
c) an alarm output in communication with the base unit for receiving the alarm signal and generating an alarm.

2. The system of claim 1, wherein exceeding the predetermined epileptiform activity level is indicative of a seizure.

3. The system of claim 1, wherein conditioning of said digital input comprises executing algorithms for providing adaptive learning functionality.

4. The system of claim 1, wherein the headset comprising 8 or fewer EEG electrodes.

5. The system of claim 4, wherein the headset comprises 4 or fewer EEG electrodes.

6. The system of claim 1, wherein the plurality of electrodes are passive, active or a combination thereof.

7. The system of claim 1, wherein the alarm is audio, visual, physical, or a combination thereof.

8. The system of claim 1, wherein the digital input is transmitted via wireless communication with the base unit.

9. The system of claim 1, wherein the base unit lacks any visual display other than to convey the alarm.

10. The system of claim 1, wherein the footprint of the base unit is 2 square feet or less.

11. The system of claim 1, wherein the headset is configured as a headband or cap.

12. The system of claim 1, wherein the headset further comprises a rechargeable power source.

13. The system of claim 1, wherein the headset further comprises one or more sensors for detecting temperature, motion, heart rate, humidity, breathing rate, blood gas concentration, or combination thereof.

14. The system of claim 13, wherein the computer-executable instructions further comprise instructions for analyzing digital input generated by the one or more additional sensors.

15. The system of claim 1, further comprising a remote monitor for receiving an alarm output.

16. The system of claim 1, wherein the base unit further comprises a port for receiving a data storage device.

17. The system of claim 15, wherein the base unit further comprises a wireless transmitter for transmitting data to the remote monitor.

18. The system of claim 15, wherein the remote monitor is a cellular telephone or PDA.

19. A method for monitoring and providing an alarm indicating that a threshold level of epileptiform activity has been exceeded in the brain of a subject, comprising:
a) collecting and responding to epileptiform data received from the subject's brain by electroencephalogram (EEG) electrodes, the collecting and responding to steps consisting essentially of:
i) collecting electrical activity from the brain of the subject via a headset comprising a plurality of 1 to 12 electrodes;
i) filtering, conditioning and analyzing a digital input generated from electrical signals collected by the plurality of EEG electrodes and transmitted from the headset to identify whether a predetermined epileptiform activity level has been exceeded;
iii) generating an alarm signal when the predetermined epileptiform activity level has been exceeded;
iv) time stamping and storing the digital signal to a data storage device when an alarm signal has been generated, thereby producing a record, wherein the record does not include unprocessed data of electrical brain activity; and
v) transmitting the alarm signal to an alarm output to generate an alarm.

20. The method of claim 19, wherein the predetermined epileptiform activity level is indicative of a seizure.

21. The method of claim 19, wherein the method further comprises analyzing data input from one or more sensors for detecting temperature, motion, heart rate, humidity, breathing rate, blood gas concentration, or combination thereof collected from the subject.

22. The method of claim 19, wherein the alarm is audio, visual, or physical.
